(19) United States
(12) Patent Application Publication
(10) Pub. No.: US 2011/0281908 A1
(43) Pub. Date: Nov. 17, 2011

(54) Aminoquinoline Derived Heat Shock Protein 90 Inhibitors, Methods Of Preparing Same, And Methods For Their Use

(75) Inventors: Aiming Sun, Atlanta, GA (US); Ganesh Thota, Decatur, GA (US); Jaeki Min, Marietta, GA (US); Pahk Thepchatri, Atlanta, GA (US); Yuhong Du, Atlanta, GA (US); James P Snyder, Atlanta, GA (US); Dennis C. Liotta, Atlanta, GA (US)

(73) Assignee: EMORY UNIVERSITY, Atlanta, GA (US)

(21) Appl. No.: 13/122,785

(22) PCT Filed: Oct. 6, 2009
(86) PCT No.: PCT/US09/59658

§ 371 Date: Aug. 2, 2011

Related U.S. Application Data
(60) Provisional application No. 61/103,152, filed on Oct. 6, 2008.

(51) Int. Cl. A61K 31/47; A61K 31/4709; C07D 401/12; C07D 215/38; A61P 35/00

(52) U.S. Cl. 514/313; 546/171; 514/311; 546/162

(57) ABSTRACT

Novel classes of molecular chaperone Heat shock protein 90 (Hsp90) inhibitors and methods for making these classes are provided herein. These compounds are useful in treating and preventing cancer and other Hsp90-related diseases, such as inflammation and neurodegenerative disorders. Also provided herein are methods of treating and preventing cancer and other Hsp90 related disease. The methods include administering to a subject a therapeutically effective amount of an Hsp90 inhibitor.

### CROSS-REFERENCE TO RELATED APPLICATIONS

This application claims priority to U.S. Provisional Application No. 61/103,152, filed Oct. 6, 2008, which is incorporated herein by reference in its entirety.

### STATEMENT REGARDING FEDERALLY FUNDED RESEARCH

This invention was made with government support under Grant No. U54 HG003918-02 awarded by the National Institutes of Health. The government has certain rights in the invention.

### BACKGROUND

Cancer refers to a class of diseases that arises from the uncontrollable growth and division of normal cells. These malignant tumors are characterized by certain characteristics, including self-sufficiency in growth signaling, insensitivity to anti-growth signaling, evasion of apoptosis, sustained angiogenesis, tissue invasion and metastasis, and limitless potential to replicate (J. Biosci. 2007, 32, 517-530). Several of the key signaling proteins of cancer cells are maintained by Heat shock protein 90 (Hsp90), which ranks amongst the most highly expressed cellular proteins. Hsp90 is a molecular chaperone with significant roles in maintaining transformation and in elevating the survival and growth potential of cancer cells. The biological role of Hsp90 is mediated by its ability to interact with client substrates such as Raf-1, Akt, Her2, cdk4 and Bcr-Abl. Hsp90 is required for the ATP-dependent refolding of denatured or “unfolded” proteins and for the conformational maturation of a subset of proteins involved in the response of cells to extracellular signals. Activation of signaling pathways mediated by these Hsp90 clients is necessary for cell proliferation, regulation of cell cycle progression and apoptosis. Additionally, gain-of-function mutations responsible for transformation often require Hsp90 for maintenance of their folded, functionally active conformations. Oncogenic transformation often enhances the tumor cell's dependency on Hsp90 function.

Hsp90 also has a significant role in the progression of neurodegenerative diseases. Many neurodegenerative diseases, such as Alzheimer's and Parkinson's diseases, are characterized by misfolded and mutated proteins. These pathogenic forms of the proteins depend upon Hsp90 for conformational stability. In addition, neurodegenerative diseases often result from deviant signaling pathways which often rely upon Hsp90 for functioning. The inhibition of this chaperone is a potential mechanism of treating these diseases. Hsp90-interfering drugs represent a class of therapeutic agents that by selective inhibition of the chaperone could exhibit a broad-range of anti-tumor activity by affecting multiple aspects of transformation regulated by Hsp90. In addition, these agents represent a potential class of drugs that promote the survival of neurons and open up a promising approach for the treatment of neurodegenerative diseases.

### SUMMARY

Novel methods and compositions for treating and preventing cancer and Hsp90 related diseases or conditions such as, for example, inflammation and neurodegenerative disorders, are provided. The methods comprise administering to a subject a therapeutically effective amount of Hsp90 inhibitor. A first class of Hsp90 inhibitors comprises compounds of the following formula:

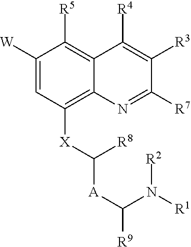

and includes pharmaceutically acceptable salts and prodrugs thereof. In this class of molecules, A is substituted or unsubstituted C₁₋₆ alkyl, substituted or unsubstituted C₂₋₆ alkenyl, or substituted or unsubstituted C₂₋₆ alkynyl; W is NHR⁶, OR⁶, or hydrogen; X is NH, S, or O; R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl, wherein R¹ and R² together are substituted or unsubstituted C₁₋₈ alkyl or substituted or unsubstituted C₂₋₈ alkenyl; R³, R⁴, and R⁵ are each independently selected from hydrogen, halogen, hydroxyl, nitro, cyano, substituted or unsubstituted amino, substituted or unsubstituted thio, substituted or unsubstituted C₁₋₁₂ alkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted C₁₋₁₂ alkylamine, substituted or unsubstituted C₂₋₁₂ alkenylamine, substituted or unsubstituted C₂₋₁₂ alkynylamine, substituted or unsubstituted arylamine, substituted or unsubstituted acyl, substituted or unsubstituted alkoxyl, and substituted or unsubstituted aryloxyl; R⁶ is hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted acyl, substituted or unsubstituted aminoalkyl, substituted or unsubstituted hydroxyalkyl, or substituted or unsubstituted alkoxyalkyl; R⁷ is hydrogen, hydroxyl, substituted or unsubstituted alkoxyl, substituted or unsubstituted amino, substituted or unsubstituted thio, substituted or unsubstituted C₁₋₁₂alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl; and R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl, wherein if X is NH, W is hydroxyl or methoxyl, and one of R¹ or R² is C₁₋₅ alkyl, the other of R¹ or R² is not hydrogen or C₁₋₅ alkyl; if X is NH and W is hydroxyl or methoxyl, R¹ and R² are not simultaneously hydrogen; and if X is NH, W is hydroxyl or methoxyl, and R⁸ is methyl, R⁹ is not hydrogen.

A second class of Hsp90 inhibitors comprises compounds of the following formula:

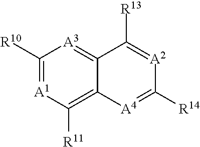

and includes pharmaceutically acceptable salts and prodrugs thereof. In this class of molecules, A¹, A², A³, and A⁴ are each independently selected from N and CR⁷, wherein R⁷ is hydrogen, substituted or unsubstituted amino, substituted or unsubstituted C₁₋₁₂ alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl, wherein at least one of A¹, A², and A³ is N; R¹⁰ is hydrogen, hydroxyl, substituted or unsubstituted alkoxyl, substituted or unsubstituted amino, substituted or unsubstituted thiol; R¹¹ is substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted amino, substituted or unsubstituted alkylsulfonylamino, substituted or unsubstituted arylsulfonylamino, substituted or unsubstituted arylalkylsulfonylamino, substituted or unsubstituted cycloalkylsulfonylamino, substituted or unsubstituted heteroalkylsulfonylamino, substituted or unsubstituted heteroarylsulfonylamino, substituted or unsubstituted heterocycloalkylsulfonylamino, substituted or unsubstituted heteroarylalkylsulfonylamino, substituted or unsubstituted cycloalkylalkylsulfonylamino, or substituted or unsubstituted heterocycloalkylalkylsulfonylamino; and R¹³ and R¹⁴ are independently hydrogen, halogen, hydroxyl, substituted or substituted amino, substituted or unsubstituted alkoxyl, substituted or unsubstituted thiol, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

A third class of Hsp90 inhibitors comprises compounds of the following formula:

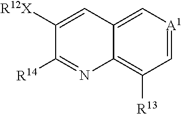

and includes pharmaceutically acceptable salts and prodrugs thereof. In this class of molecules, A¹ is CH or N; X is NH, S, or O; R¹² is hydrogen or substituted or unsubstituted alkyl; and R¹³ and R¹⁴ are independently hydrogen, substituted or substituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl, wherein R¹³ and R¹⁴ are not both hydrogen.

A fourth class of Hsp90 inhibitors comprises compounds of the following formula:

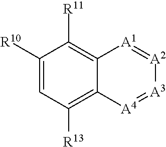

or a pharmaceutically acceptable salt or prodrug thereof, wherein A¹, A², A³, and A⁴ are each independently selected from N and CR⁷, wherein each R⁷ is independently hydrogen or substituted or unsubstituted amino; R¹⁰ is hydrogen, hydroxyl, substituted or unsubstituted alkoxyl, substituted or unsubstituted amino, substituted or unsubstituted carbamate, or substituted or unsubstituted thiol; R¹¹ is hydroxyl, substituted or unsubstituted alkoxyl, substituted or unsubstituted amino, substituted or unsubstituted carbamate, substituted or unsubstituted thiol, or substituted or unsubstituted acyl; and R¹³ is substituted or unsubstituted amino.

A fifth class of Hsp90 inhibitors comprises compounds of the following formula:

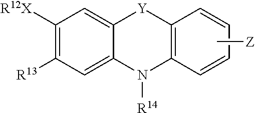

and includes pharmaceutically acceptable salts and prodrugs thereof. In this class of molecules, Y is NR¹, O, S, CHR¹, or CHR¹—CHR², wherein R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl; Z is hydrogen or halogen; X is NH, S, or O; R¹² is hydrogen or substituted or unsubstituted alkyl; R¹³ is hydrogen, substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl; and R¹⁴ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl, wherein R¹³ and R¹⁴ are not both hydrogen.

A sixth class of Hsp90 inhibitors comprises compounds of the following formula:

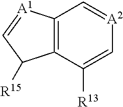

and includes pharmaceutically acceptable salts and prodrugs thereof. In this class of molecules, A¹ and A² are each independently selected from CH and N; R¹³ is substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl; and R¹⁵ is hydrogen, hydroxyl, or substituted or unsubstituted alkoxyl.

A seventh class of Hsp90 inhibitors comprises compounds of the following formula:

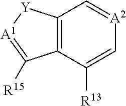

and includes pharmaceutically acceptable salts and prodrugs thereof. In this class of molecules, X is NH, S, or O; Y is NR¹, O, S, and CHR¹, wherein R¹ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl; A¹ and A² are each independently selected from CH and N; R¹³ is substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl; and R¹⁵ is hydrogen, hydroxyl, or substituted or unsubstituted alkoxyl.

An eighth class of Hsp90 inhibitors comprises compounds of the following formula:

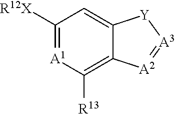

and includes pharmaceutically acceptable salts and prodrugs thereof. In this class of molecules, X is NH, S, or O; Y is NR¹, O, S, and CHR¹, wherein R¹ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl; A¹, A², and A³ are each independently selected from CH and N; R¹³ is substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl; and R¹² is hydrogen or substituted or unsubstituted alkyl.

A ninth class of Hsp90 inhibitors comprises compounds of the following formula:

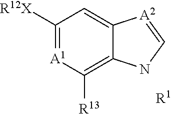

and includes pharmaceutically acceptable salts and prodrugs thereof. In this class of molecules, X is NH, S, or O; A¹ and A² are each independently selected from CH and N; R¹ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl; R¹² is hydrogen or substituted or unsubstituted alkyl; and R¹³ is substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

A tenth class of Hsp90 inhibitors comprises compounds of the following formula:

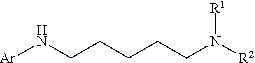

or a pharmaceutically acceptable salt or prodrug thereof, wherein Ar is substituted or unsubstituted aryl or substituted or unsubstituted heteroaryl; and R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl.

Also provided is a method of preparing compounds for use as Hsp90 inhibitors. The method of preparing compounds comprises the following steps: treating 6-methoxy-8-amino-quinoline with excess bromoalkylnitrile in solvent to produce a 4-(6-methoxyquinolin-8-ylamino)alkylnitrile; reducing the 4-(6-methoxyquinolin-8-ylamino) alkylnitrile with a hydride donor to produce a 4-(6-methoxyquinolin-8-ylamino)alkylamine; and alkylating the 4-(6-methoxyquinolin-8-ylamino)alkylamino to produce a N¹,N¹-dialkyl-N⁴-(6-methoxyquinolin-8-yl)alkyldiamine. In some embodiments, the method further comprises the step of treating the N¹,N¹-dialkyl-N⁴-(6-methoxyquinolin-8-yl)alkyldiamine with hydrobromic acid to produce a N¹,N¹-dialkyl-N⁴-(6-hydroxyquinolin-8-yl)alkyldiamine. In some embodiments, the hydride donor in the reducing step is lithium aluminum hydride. In some embodiments, the solvent in the treating step is a mixture of triethylamine and methanol.

The details of one or more embodiments are set forth in the description below. Other features, objects, and advantages will be apparent from the description and from the claims.

### DESCRIPTION OF DRAWINGS

FIG. 1 is a graph showing the dose response binding inhibition of compound 1 in a competitive binding assay.

### DETAILED DESCRIPTION

The term “comprising” and variations thereof as used herein are used synonymously with the term “including” and variations thereof and are open, non-limiting terms.

Novel classes of Hsp90 inhibitors are provided herein. These compounds are useful in treating, preventing, and/or ameliorating cancer and other Hsp90 associated diseases or conditions such as, for example, inflammation and neurodegenerative disorders. Specifically, pharmaceutically acceptable salts, prodrugs, and derivatives of aminoquinoline compounds are provided. Methods of their synthesis and use in the treatment of Hsp90 associated conditions are also provided.

A first group of Hsp90 inhibitors comprises compounds represented by Formula I:

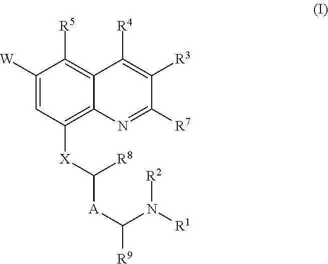

or a pharmaceutically acceptable salt or prodrug thereof. In some examples, Formula I is a hydrobromide salt.

In Formula I, A is substituted or unsubstituted C₁₋₆ alkyl, substituted or unsubstituted C₂₋₆ alkenyl, or substituted or unsubstituted C₂₋₆ alkynyl.

Also, in Formula I, W is hydrogen, NHR⁶, or OR⁶ wherein R⁶ is hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted acyl, substituted or unsubstituted aminoalkyl, substituted or unsubstituted hydroxyalkyl, or substituted or unsubstituted alkoxyalkyl.

Additionally, in Formula I, X is NH, S, or O. In some examples, X is NH.

Further, in Formula I, R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl. In some examples, R¹ and R² together form substituted or unsubstituted C₁₋₈ alkyl or substituted or unsubstituted C₂₋₈ alkenyl.

Also, in Formula I, R³, R⁴, and R⁵ are each independently selected from hydrogen, halogen, hydroxyl, nitro, cyano, substituted or unsubstituted amino, substituted or unsubstituted thio, substituted or unsubstituted C₁₋₁₂ alkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted C₁₋₁₂ alkylamine, substituted or unsubstituted C₂₋₁₂ alkenylamine, substituted or unsubstituted C₂₋₁₂ alkynylamine, substituted or unsubstituted arylamine, substituted or unsubstituted acyl, substituted or unsubstituted alkoxyl, and substituted or unsubstituted aryloxyl. In some examples, R⁵ is substituted or unsubstituted amido.

Further, in Formula I, R⁷ is hydrogen, hydroxyl, substituted or unsubstituted alkoxyl, substituted or unsubstituted amino, substituted or unsubstituted thio, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl. In some examples, R⁷ is substituted or unsubstituted amino.

Furthermore, in Formula I, R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl.

In some examples of Formula I, if X is NH, W is hydroxyl or methoxyl, and one of R¹ or R² is C₁₋₅ alkyl, then the other of R¹ or R² is not hydrogen or C₁₋₅ alkyl.

Additionally, in some examples of Formula I, if X is NH and W is hydroxyl or methoxyl, then R¹ and R² are not simultaneously hydrogen.

Also, in some examples of Formula I, if X is NH, W is hydroxyl or methoxyl, and R⁸ is methyl, then R⁹ is not hydrogen.

An example of Formula I includes the following:

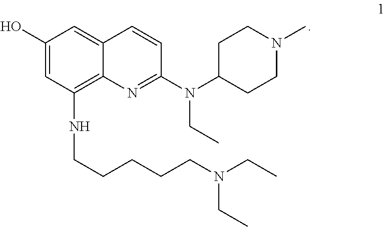

In some examples, Formula I can be Formula I-A:

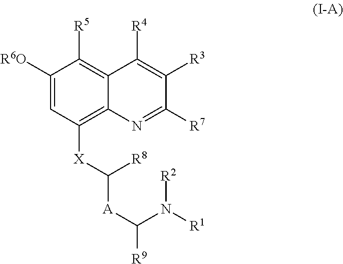

or a pharmaceutically acceptable salt or prodrug thereof. In some examples, Formula I-A is a hydrobromide salt.

In Formula I-A, A is substituted or unsubstituted C₁₋₆ alkyl, substituted or unsubstituted C₂₋₆ alkenyl, or substituted or unsubstituted C₂₋₆ alkynyl.

Also, in Formula I-A, X is S or O.

Additionally, in Formula I-A, R¹, R², and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl.

In some examples, R¹ is hydrogen, methyl, ethyl, propyl, butyl, pentyl, isopropyl, sec-butyl, isobutyl, benzyl, benzoyl, acetyl, or substituted or unsubstituted sulfonyl (e.g., SO₂R⁸). In some examples, R² is hydrogen, methyl, ethyl, propyl, butyl, pentyl, isopropyl, sec-butyl, isobutyl, benzyl, benzoyl, acetyl, or

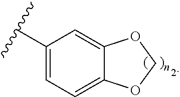

In some examples, R¹ and R² together form substituted or unsubstituted C₁₋₈ alkyl or substituted or unsubstituted C₂₋₈ alkenyl. For example, R¹—N—R² can combine to form aziridine, azetidine, pyrrolidine, or piperidine. In some examples, R⁷ is hydrogen, methyl, ethyl, propyl, isopropyl, butyl, isobutyl, t-butyl, acetyl, or propanoyl.

Further, in Formula I-A, R³, R⁴, and R⁵ are each independently selected from hydrogen, halogen, hydroxyl, nitro, substituted or unsubstituted amino, cyano, substituted or unsubstituted C₁₋₁₂ alkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted C₁₋₁₂ alkylamine, substituted or unsubstituted C₂₋₁₂ alkenylamine, substituted or unsubstituted C₂₋₁₂ alkynylamine, substituted or unsubstituted arylamine, substituted or unsubstituted alkoxyl, and substituted or unsubstituted aryloxyl.

In some examples, R³ is hydrogen, methyl, ethyl, propyl, or NH-ethyl. In some examples, R⁴ is hydrogen, methyl, ethyl, propyl, NH-ethyl, NH-phenyl, chloro, hydroxyl, methoxyl, or phenoxyl. In some examples, R⁵ is hydrogen, hydroxyl, chloro, nitro, amino, NH-phenyl, p-chlorophenyl, or p-methoxyphenyl.

Additionally, in Formula I-A, R⁶ is hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted acyl, substituted or unsubstituted aminoalkyl, substituted or unsubstituted hydroxyalkyl, or substituted or unsubstituted alkoxyalkyl. In some examples, R⁶ is hydrogen, methyl, ethyl, allyl, acetyl, or —(CH₂)n₁T, wherein n₁ is 1-5 and T is hydrogen, hydroxyl, amino, or alkoxyl.

Furthermore, in Formula I-A, R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl. In some examples, one or both of R⁸ and R⁹ is hydrogen, methyl, or ethyl.

In some examples, Formula I can be Formula I-B:

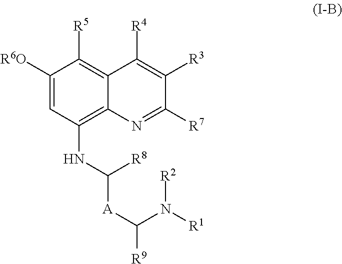

or a pharmaceutically acceptable salt or prodrug thereof. In some examples, Formula I-B is a hydrobromide salt.

In Formula I-B, A is substituted or unsubstituted C₁₋₆ alkyl, substituted or unsubstituted C₂₋₆ alkenyl, or substituted or unsubstituted C₂₋₆ alkynyl.

Also, in Formula I-B, R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl. In some examples, R¹ and R² are not C₁₋₁₂ alkyl. In some examples, R¹ is hydrogen, benzyl, benzoyl, acetyl, or substituted or unsubstituted sulfonyl (e.g., SO₂R⁸). In some examples, R² is hydrogen, benzyl, benzoyl, acetyl, or

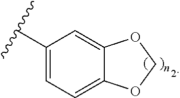

In some examples, R¹ and R² together form substituted or unsubstituted C₁₋₈ alkyl or substituted or unsubstituted C₂₋₈ alkenyl. For example, R¹—N—R² can combine to form aziridine, azetidine, pyrrolidine, or piperidine.

Additionally, in Formula I-B, R³, R⁴, and R⁵ are each independently selected from hydrogen, halogen, hydroxyl, nitro, substituted or unsubstituted amino, cyano, substituted or unsubstituted C₁₋₁₂ alkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted C₁₋₁₂ alkylamine, substituted or unsubstituted C₂₋₁₂ alkenylamine, substituted or unsubstituted C₂₋₁₂ alkynylamine, substituted or unsubstituted arylamine, substituted or unsubstituted alkoxyl, and substituted or unsubstituted aryloxyl.

In some examples, R³ is hydrogen, methyl, ethyl, propyl, or NH-ethyl. In some examples, R⁴ is hydrogen, methyl, ethyl, propyl, NH-ethyl, NH-phenyl, chloro, hydroxyl, methoxyl, or phenoxyl. In some examples, R⁵ is hydrogen, hydroxyl, chloro, nitro, amino, NH-phenyl, p-chlorophenyl, or p-methoxyphenyl.

Further, in Formula I-B, R⁶ is hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted acyl, substituted or unsubstituted aminoalkyl, substituted or unsubstituted hydroxyalkyl, or substituted or unsubstituted alkoxyalkyl. In some examples, R⁶ is hydrogen, methyl, ethyl, allyl, acetyl, or —(CH₂)n₁T, wherein n₁ is 1-5 and T is hydrogen, hydroxyl, amino, or alkoxyl.

Also, in Formula I, R⁷ is hydrogen, substituted or unsubstituted C₁₋₁₂ alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl. In some examples, R⁷ is hydrogen, methyl, ethyl, propyl, isopropyl, butyl, isobutyl, t-butyl, acetyl, or propanoyl.

Further, in Formula I-B, R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl. In some examples, one or both of R⁸ and R⁹ is hydrogen, methyl, or ethyl.

In some examples of Formula I-B, if R⁶ is hydrogen or methyl and one of R¹ or R² is C₁₋₅ alkyl, then the other of R¹ or R² is not hydrogen or C₁₋₅ alkyl. In some examples of Formula I, if R⁶ is hydrogen or methyl, then R¹ and R² are not simultaneously hydrogen. Also, in some examples of Formula I-B, if R⁶ is hydrogen or methyl and R⁸ is methyl, then R⁹ is not hydrogen.

Examples of Formula I include the following:

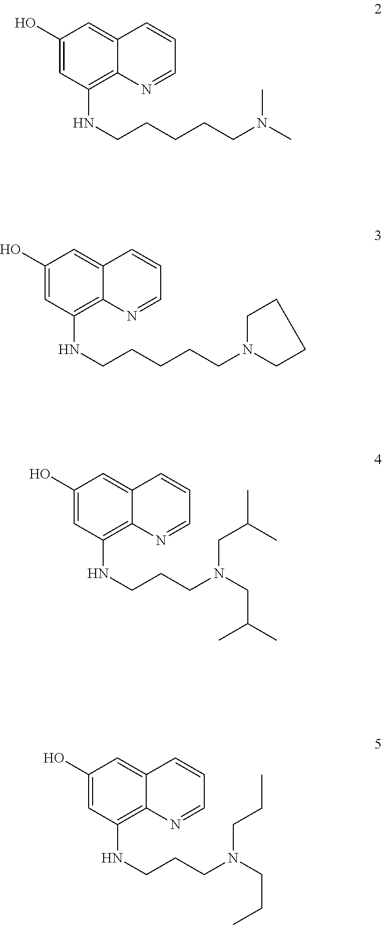

A second group of Hsp90 inhibitors comprises compounds represented by Formula II:

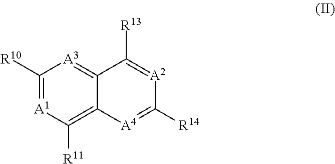

or a pharmaceutically acceptable salt or prodrug thereof.

In Formula II, A¹, A², A³, and A⁴ are each independently selected from N and CR⁷, wherein at least one of A¹, A², and A³ is N. In some examples, A³ is N.

Also, in Formula II, R⁷ is hydrogen, substituted or unsubstituted amino, substituted or unsubstituted C₁₋₁₂ alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl.

Additionally, in Formula II, R¹⁰ is hydrogen, hydroxyl, substituted or unsubstituted alkoxyl, substituted or unsubstituted amino, or substituted or unsubstituted thiol.

Further, in Formula II, R¹¹ is substituted or unsubstituted C₁₋₁₂ alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted amino, substituted or unsubstituted alkylsulfonylamino, substituted or unsubstituted arylsulfonylamino, substituted or unsubstituted arylalkylsulfonylamino, substituted or unsubstituted cycloalkylsulfonylamino, substituted or unsubstituted heteroalkylsulfonylamino, substituted or unsubstituted heteroarylsulfonylamino, substituted or unsubstituted heterocycloalkylsulfonylamino, substituted or unsubstituted heteroarylalkylsulfonylamino, substituted or unsubstituted cycloalkylalkylsulfonylamino, or substituted or unsubstituted heterocycloalkylalkylsulfonylamino.

Further, in Formula II, R¹³ and R¹⁴ are independently hydrogen, halogen, hydroxyl, substituted or substituted amino, substituted or unsubstituted alkoxyl, substituted or unsubstituted thiol, substituted or unsubstituted C₁₋₁₂ alkyl, substituted or unsubstituted C₁₋₁₂haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

In some examples, Formula II can be Formula II-A:

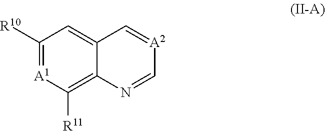

or a pharmaceutically acceptable salt or prodrug thereof.

In Formula II-A, A¹ and A² are each independently selected from CH and N.

Also, in Formula II-A, R¹⁰ is hydrogen, hydroxyl, substituted or unsubstituted alkoxyl, substituted or unsubstituted amino, or substituted or unsubstituted thiol. In some examples, R¹⁰ is substituted or unsubstituted amino.

Additionally, in Formula II-A, R¹¹ is substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted amino, substituted or unsubstituted alkylsulfonylamino, substituted or unsubstituted arylsulfonylamino, substituted or unsubstituted arylalkylsulfonylamino, substituted or unsubstituted cycloalkylsulfonylamino, substituted or unsubstituted heteroalkylsulfonylamino, substituted or unsubstituted heteroarylsulfonylamino, substituted or unsubstituted heterocycloalkylsulfonylamino, substituted or unsubstituted heteroarylalkylsulfonylamino, substituted or unsubstituted cycloalkylalkylsulfonylamino, or substituted or unsubstituted heterocycloalkylalkylsulfonylamino. In some examples, R¹¹ is alkyl, alkenyl, alkynyl, cycloalkyl, aryl, or unsubstituted or unsubstituted amino.

Examples of Formula II-A include the following:

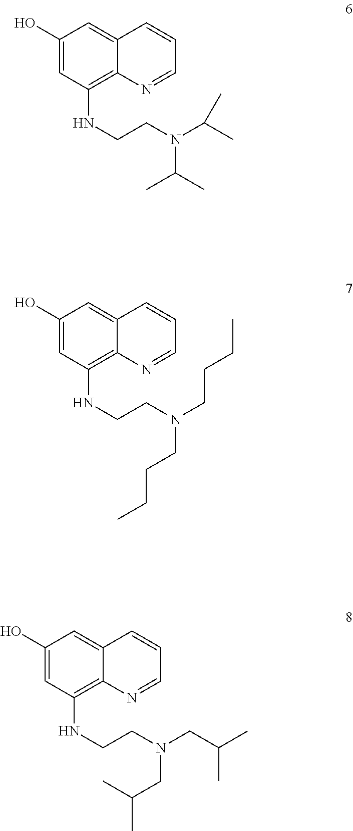

In some examples, Formula II can be Formula II-B:

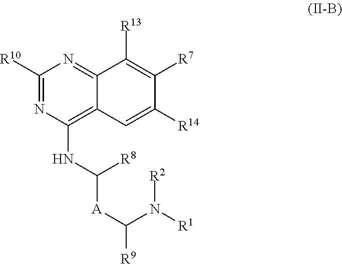

or a pharmaceutically acceptable salt or prodrug thereof.

In Formula II-B, R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl. In some examples, R¹ and R² together form substituted or unsubstituted C₁₋₈ alkyl or substituted or unsubstituted C₂₋₈ alkenyl.

Also, in Formula II-B, R² is hydrogen, substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl.

Additionally, in Formula II-B, R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl.

Further, in Formula II-B, R¹⁰ is hydrogen, hydroxyl, substituted or unsubstituted alkoxyl, substituted or unsubstituted amino, or substituted or unsubstituted thiol.

Also, in Formula II-B, R¹¹ is substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted amino, substituted or unsubstituted alkylsulfonylamino, substituted or unsubstituted arylsulfonylamino, substituted or unsubstituted arylalkylsulfonylamino, substituted or unsubstituted cycloalkylsulfonylamino, substituted or unsubstituted heteroalkylsulfonylamino, substituted or unsubstituted heteroarylsulfonylamino, substituted or unsubstituted heterocycloalkylsulfonylamino, substituted or unsubstituted heteroarylalkylsulfonylamino, substituted or unsubstituted cycloalkylalkylsulfonylamino, or substituted or unsubstituted heterocycloalkylalkylsulfonylamino.

Further, in Formula II-B, R¹³ and R¹⁴ are each independently hydrogen, halogen, substituted or substituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

In some examples, Formula II can be Formula II-C:

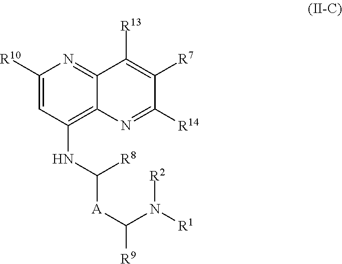

or a pharmaceutically acceptable salt or prodrug thereof.

In Formula II-C, R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl. In some examples, R¹ and R² together form substituted or unsubstituted C₁₋₈ alkyl or substituted or unsubstituted C₂₋₈ alkenyl.

Also, in Formula II-C, R⁷ is hydrogen, substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl.

Additionally, in Formula II-C, R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl.

Further, in Formula II-C, R¹⁰ is hydrogen, hydroxyl, substituted or unsubstituted alkoxyl, substituted or unsubstituted amino, or substituted or unsubstituted thiol. In some examples, R¹⁰ is substituted or unsubstituted amino.

Also, in Formula II-C, R¹¹ is substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted amino, substituted or unsubstituted alkylsulfonylamino, substituted or unsubstituted arylsulfonylamino, substituted or unsubstituted arylalkylsulfonylamino, substituted or unsubstituted cycloalkylsulfonylamino, substituted or unsubstituted heteroalkylsulfonylamino, substituted or unsubstituted heteroarylsulfonylamino, substituted or unsubstituted heterocycloalkylsulfonylamino, substituted or unsubstituted heteroarylalkylsulfonylamino, substituted or unsubstituted cycloalkylalkylsulfonylamino, or substituted or unsubstituted heterocycloalkylalkylsulfonylamino.

Further, in Formula II-C, R¹³ and R¹⁴ are independently hydrogen, halogen, substituted or substituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

In some examples of Formula II-C, A is (CH₂)₃, R⁸ is hydrogen, and R⁹ is hydrogen.

A third group of Hsp90 inhibitors comprises compounds represented by Formula III:

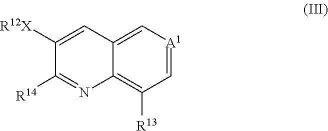

or pharmaceutically acceptable salts or prodrugs thereof.

In Formula III, A¹ is CH and N.

Also, in Formula III, X is NH, S, or O.

Additionally, in Formula III, R¹² is hydrogen or substituted or unsubstituted C₁₋₁₂ alkyl. In some examples, R¹²X is substituted or unsubstituted amino.

Further, in Formula III, R¹³ and R¹⁴ are independently hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl, wherein R¹³ and R¹⁴ are not both hydrogen. In some examples, R¹³ or R¹⁴ is substituted or unsubstituted amino.

A fourth group of Hsp90 inhibitors comprises compounds represented by Formula IV:

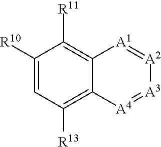

or a pharmaceutically acceptable salt or prodrug thereof.

In Formula IV, A¹, A², A³, and A⁴ are each independently selected from N and CR⁷. If one or more of R⁷ is present in Formula IV, each R⁷ is independently hydrogen, substituted or unsubstituted C₁₋₈ alkyl, substituted or unsubstituted C₂₋₈ alkenyl, or substituted or unsubstituted amino. In some examples, R⁷ is hydrogen.

In some examples, A3 is CR⁷. In some examples, A¹, A², A³, and A⁴ are CR⁷. Adjacent R⁷ groups can combine to form a substituted or unsubstituted cycloalkyl, a substituted or unsubstituted heterocycloalkyl, a substituted or unsubstituted aryl, or a substituted or unsubstituted heteroaryl.

Also, in Formula IV, R¹⁰ is hydrogen, hydroxyl, substituted or unsubstituted alkoxyl, substituted or unsubstituted amino, substituted or unsubstituted carbamate, or substituted or unsubstituted thiol.

Additionally, in Formula IV, R¹¹ is hydroxyl, substituted or unsubstituted alkoxyl, substituted or unsubstituted amino, substituted or unsubstituted carbamate, substituted or unsubstituted thiol, or substituted or unsubstituted acyl. In some examples, R¹¹ is amido.

Further, in Formula IV, R¹³ is substituted or unsubstituted amino

A fifth group of Hsp90 inhibitors comprises compounds represented by Formula V:

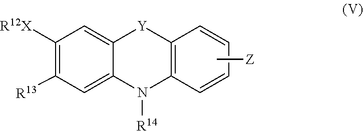

or pharmaceutically acceptable salts or prodrugs thereof.

In Formula V, Y is NR¹, O, S, CHR¹, or CHR¹—CHR², wherein R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl. In some examples, Y is NR¹, O, S, or CHR¹.

Also, in Formula V, Z is hydrogen or a halogen.

Additionally, in Formula V, X is NH, S, or O.

Also, in Formula V, R¹² is hydrogen or substituted or unsubstituted alkyl.

Further, in Formula V, R¹³ is hydrogen, substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl.

Furthermore, in Formula V, R¹⁴ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl. R¹³ and R¹⁴ are not both hydrogen.

A sixth group of Hsp90 inhibitors comprises compounds represented by Formula VI:

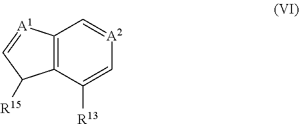

or pharmaceutically acceptable salts or prodrugs thereof.

In Formula VI, A¹ and A² are each independently selected from CH and N.

Also, in Formula VI, R¹³ is substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

Additionally, in Formula VI, R¹⁵ is hydrogen, hydroxyl, or substituted or unsubstituted alkoxyl.

A seventh group of Hsp90 inhibitors comprises compounds represented by Formula VII:

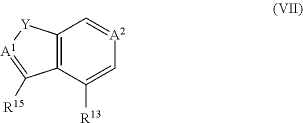

or pharmaceutically acceptable salts or prodrugs thereof.

In Formula VII, X is NH, S, or O.

Also, in Formula VII, Y is NR¹, O, S, or CHR¹, wherein R¹ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl.

Additionally, in Formula VII, A¹ and A² are each independently selected from CH and N.

Also, in Formula VII, R¹³ is substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, or substituted or unsubstituted heterocycloalkylalkyl.

Further, in Formula VII, R¹⁵ is hydrogen, hydroxyl, or substituted or unsubstituted alkoxyl.

An eighth group of Hsp90 inhibitors comprises compounds represented by Formula VIII:

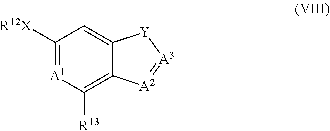

or pharmaceutically acceptable salts or prodrugs thereof.

In Formula VIII, X is NH, S, or O.

Also, in Formula VIII, Y is NR¹, O, S, or CHR¹, wherein R¹ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl.

Additionally, in Formula VIII, A¹, A², and A³ are each independently selected from CH and N.

Also, in Formula VIII, R¹² is hydrogen or substituted or unsubstituted alkyl.

Further, in Formula VIII, R¹³ is substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

A ninth group of Hsp90 inhibitors comprises compounds represented by Formula IX:

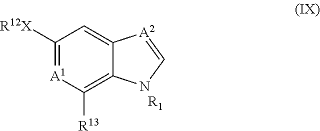

or pharmaceutically acceptable salts or prodrugs thereof.

In Formula IX, X is NH, S, or O.

Also, in Formula IX, A¹ and A² are each independently selected from CH and N.

Additionally, in Formula IX, R¹ is hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl.

Also, in Formula IX, R¹² is hydrogen or substituted or unsubstituted alkyl.

Further, in Formula IX, R¹³ is substituted or unsubstituted amino, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, and substituted or unsubstituted heterocycloalkylalkyl.

In some examples of Formulas III-IX, R¹¹ or R¹³ can independently be alkyl, alkenyl, alkynyl, cycloalkyl, or aryl. In some examples of Formulas III-IX, R¹³ is substituted or unsubstituted amino.

In other examples of Formulas III-IX, R¹¹ or R¹³ can independently be

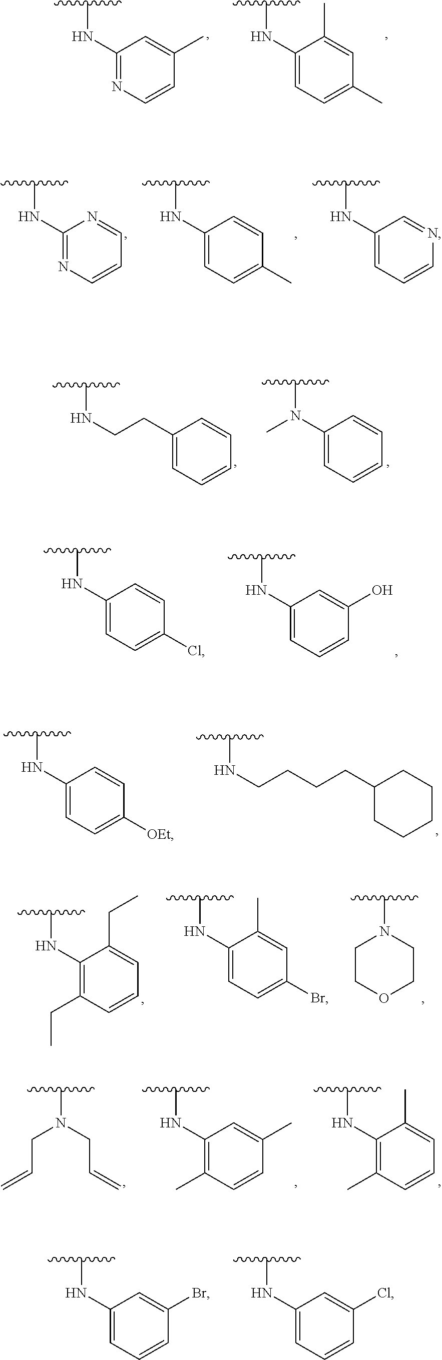

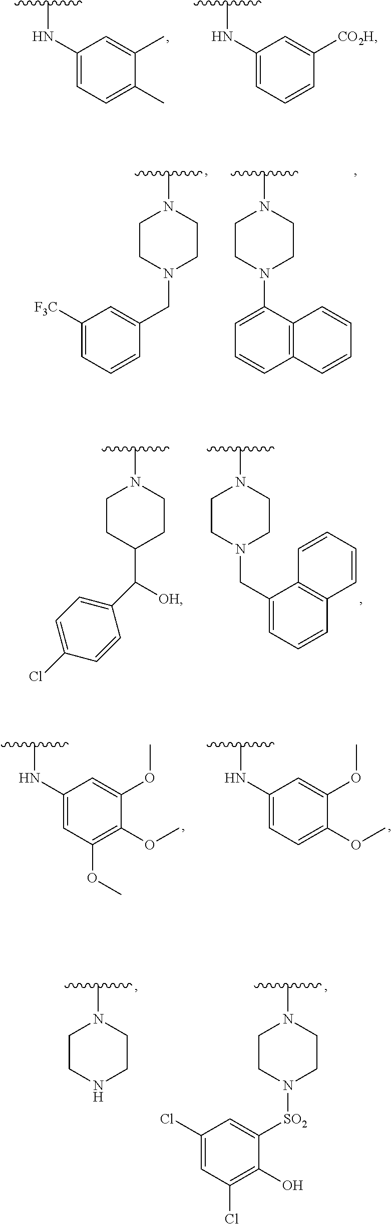

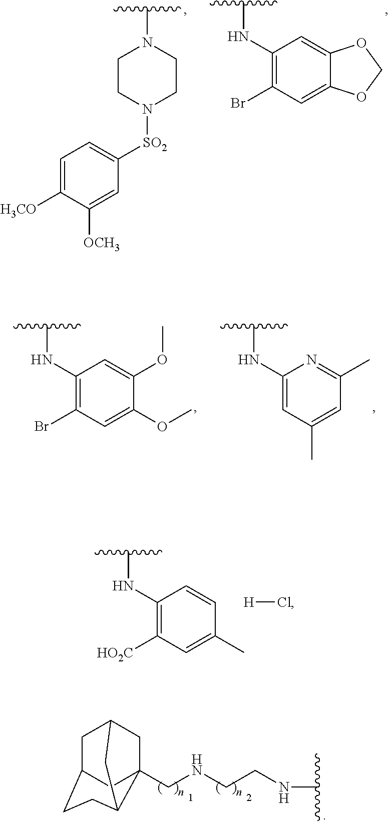

A tenth group of Hsp90 inhibitors comprises compounds represented by Formula X:

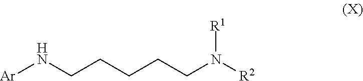

or a pharmaceutically acceptable salt or prodrug thereof.

In Formula X, Ar is substituted or unsubstituted aryl or substituted or unsubstituted heteroaryl. In some examples, Ar is substituted or unsubstituted naphthalene, substituted or unsubstituted quinoline, substituted or unsubstituted quinazoline, or substituted or unsubstituted naphthyridine.

Also, in Formula X, R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl. In some examples, R¹ and R² is substituted or unsubstituted C₁₋₆ alkyl.

Examples of Hsp90 inhibitors represented by Formulas I, II, III, IV, V, VI, VII, VIII, IX, and X are provided below:

| No | Compound |
|---|---|
| 9 | 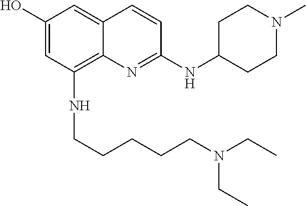 |
| 10 | 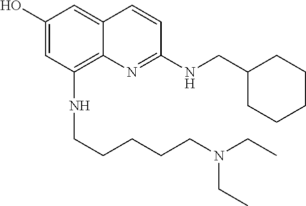 |
| 11 | 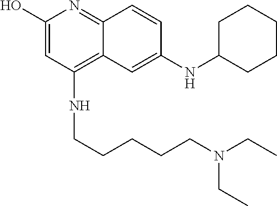 |
| 12 | 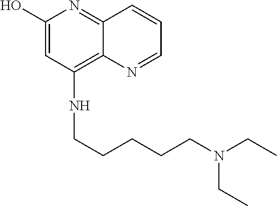 |
| 13 | 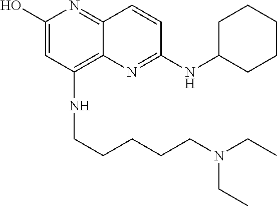 |
| 14 | 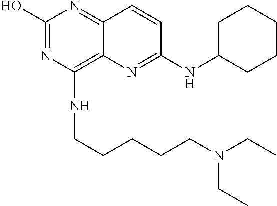 |
| 15 | 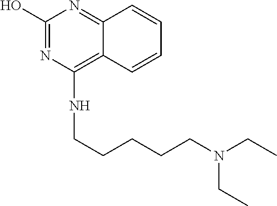 |
| 16 | 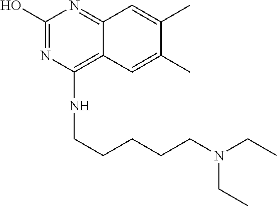 |
| 17 | 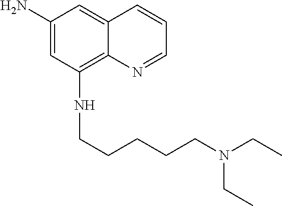 |
| 18 | 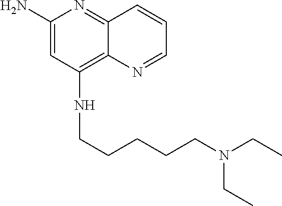 |
| 19 | 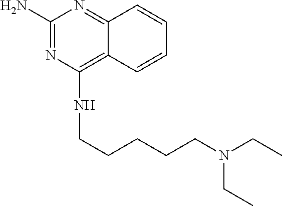 |
| 20 | 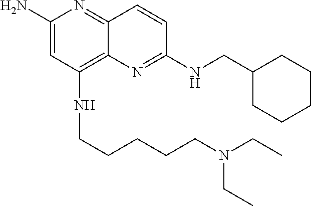 |
| 21 | 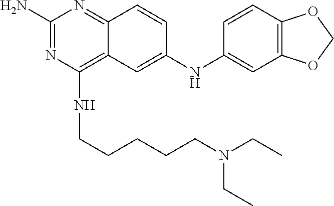 |
| 22 | 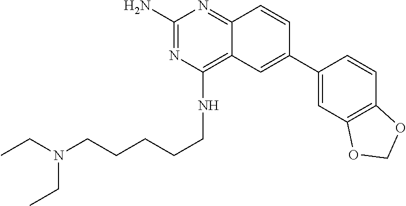 |
| 23 | 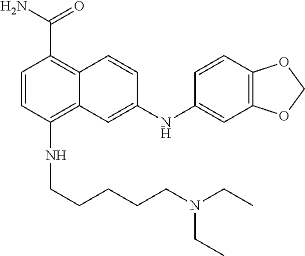 |
| 24 | 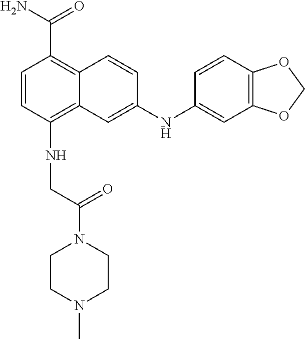 |
| 25 | 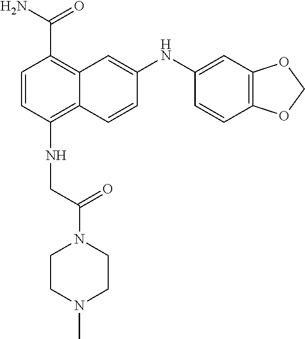 |
| 26 | 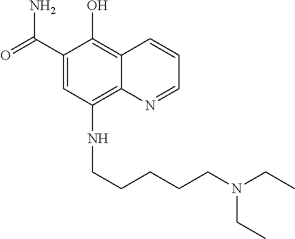 |
| 27 | 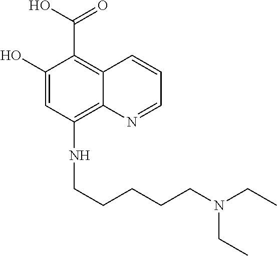 |
| 28 | 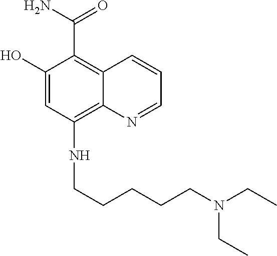 |
| 29 | 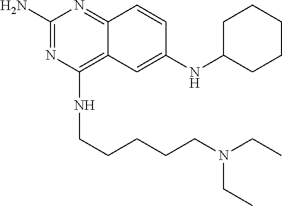 |
| 30 | 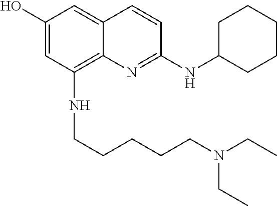 |
| 31 | 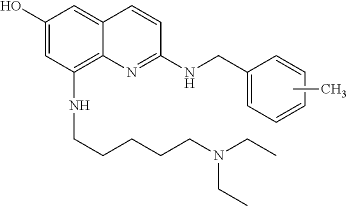 |
| 32 | 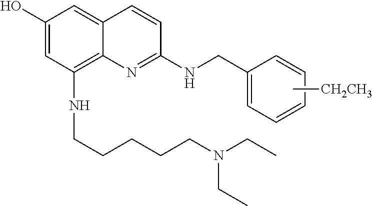 |
| 33 | 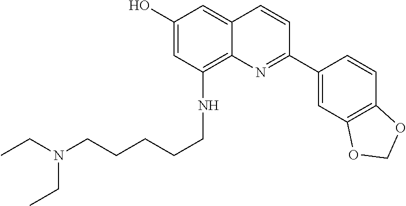 |
| 34 | 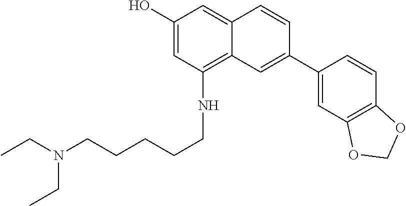 |
| 35 | 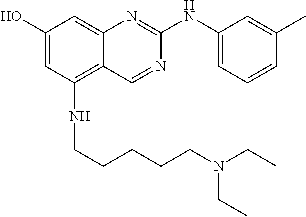 |
| 36 | 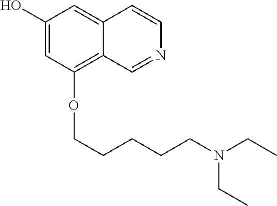 |
| 37 | 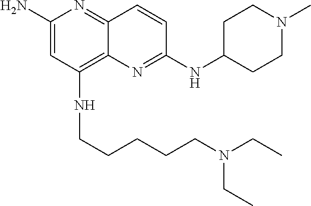 |
| 38 | 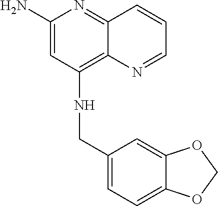 |
| 39 | 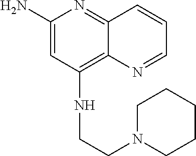 |
| 40 | 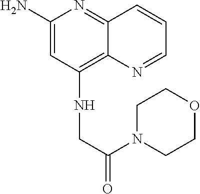 |
| 41 | 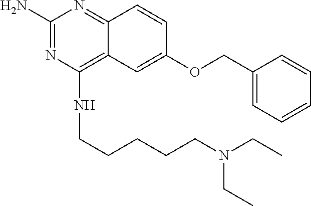 |
| 42 | 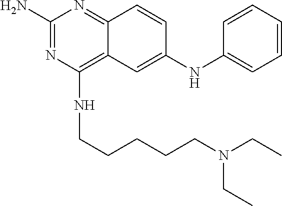 |
| 43 | 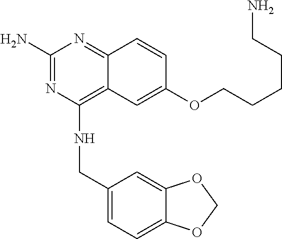 |
| 44 | 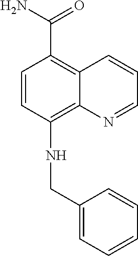 |
| 45 | 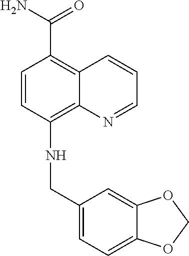 |
| 46 | 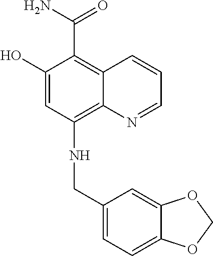 |
| 47 | 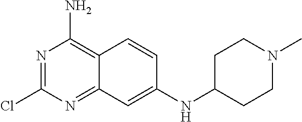 |
| 48 | 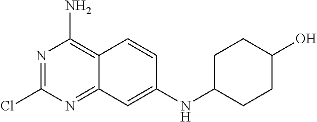 |

Variations on Formulas I, II, III, IV, V, VI, VII, VIII, IX, and X include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety. The synthesis and subsequent testing of various compounds as described for Formulas I, II, III, IV, V, VI, VII, VIII, IX, and X to determine efficacy is contemplated.

As used herein, the terms “alkyl”, “alkenyl”, “alkynyl”, and “cycloalkyl” can include straight-chain and branched monovalent substituents. Examples include methyl, ethyl, isobutyl, cyclohexyl, cyclopentylethyl, 2-propenyl, 3-butynyl, and the like. “Heteroalkyl”, “heteroalkenyl”, “heteroalkynyl”, and “heterocycloalkyl” refer to compounds that are similar to “alkyl”, “alkenyl”, “alkynyl”, and “cycloalkyl” but that further contain a hetero atom such as O, S, or N or combinations thereof. The terms “cycloalkylalkyl” and “heterocycloalkylalkyl” refer to cycloalkyl and heterocycloalkyl groups that are bonded to alkyl. The term “aryl” can include monocyclic or fused bicyclic moieties such as phenyl or naphthyl and the term “heteroaryl” can include monocyclic or fused bicyclic ring systems containing one or more heteroatoms selected from O, S, and N. Heteroaryls can include, for example, 5-, 6-, 7-, and 8-membered rings. Thus, aryl and heteroaryl systems can include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Similarly, the terms “arylalkyl” and “heteroarylalkyl” refer to aryl and heteroaryl groups bonded to alkyl groups, including substituted or unsubstituted, saturated or unsaturated, carbon chains. The term “substituted” indicates the main substituent has attached to it one or more additional components, such as, for example, OH, halogen, or one of the other substituents listed above. Substituted aryls can include, for example, monosubstituted, disubstituted, or trisubstituted aryls. As used herein, the term “acyl” indicated a substituted carbonyl. Acyls can include, for example, ketones, aldehydes, amides, carboxylic acids, esters, acyl halides, acid anhydrides, and the like.

A general method of preparing a compound of Formula I includes treating a compound of Formula XI:

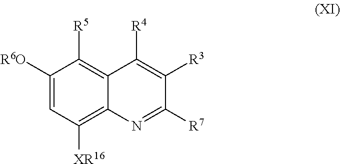

wherein X is NH, S, or O and R¹⁶ is H, with excess bromoalkylnitrile in solvent to produce the compound of formula XI, wherein R¹⁶ is alkylnitrile. The compounds of formula XI, wherein R¹⁶ is alkylnitrile, can then be reduced with a hydride donor to produce the compound of formula XI, wherein R¹⁶ is alkylamine. An example of a hydride donor that can be used in the reduction step is lithium aluminum hydride. The compound of formula XI, wherein R¹⁶ is alkylamine can then be alkylated to produce the compound of formula XI, wherein R¹⁶ is N¹,N¹-dialkylamine. This compound can then be treated with hydrobromic acid in solvent to produce the compound of formula XI, wherein R¹⁶ is N¹,N¹-dialkylamine and R⁶ is H. The solvent used in the treatment step can be, for example, a mixture of triethylamine and methanol.

The aminoquinoline compounds, for example, can be prepared by refluxing 6-methoxy-8-amino-quinoline and 4-bromobutanenitrile in a mixture of triethylamine and methanol. The mixture can then be concentrated and purified by chromatography to afford the desired nitrile. A solution of nitrile in THF can then be reacted with lithium aluminum hydride. The reaction can be quenched by the addition of NaHCO₃ and worked up under normal conditions. The crude product can then be dissolved in dichloromethane and acetaldehyde added. Then, NaBH(OAc)₃ can be added and the product can be stirred to allow the reaction to occur. After the reaction is complete, silica gel chromatography provides the desired, purified diamine. A solution of the diamine in HBr can be reacted in a microwave initiator. The resulting product can then be purified by chromatography to give the desired aminoquinoline.

Compound 1 can be synthesized according to the method shown in the following scheme:

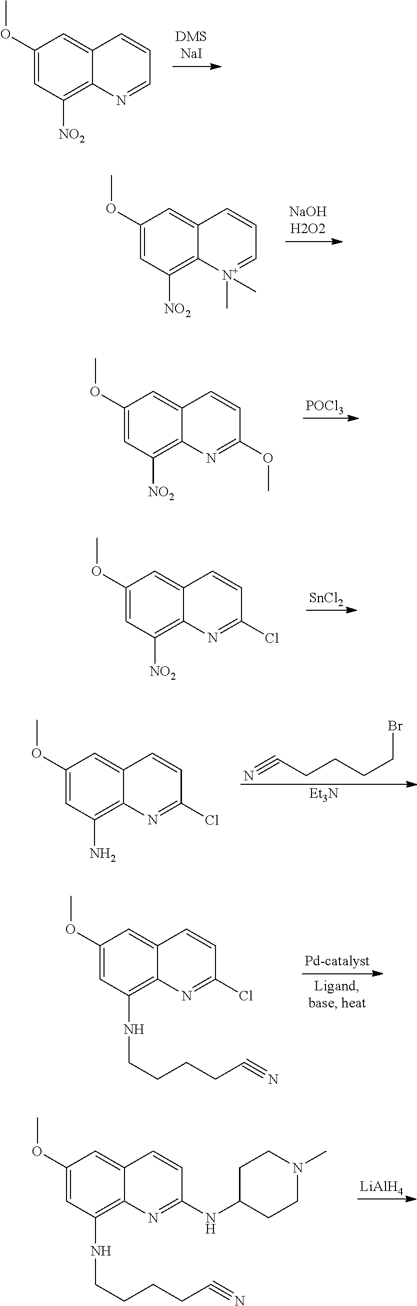

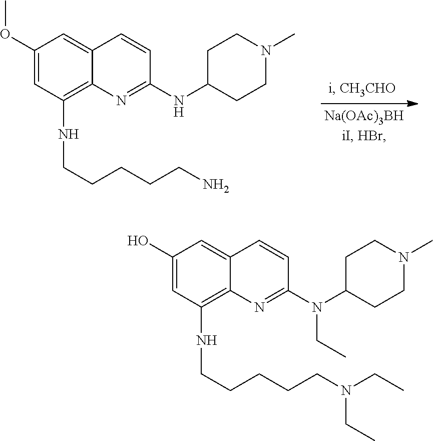

A general method of preparing a compound of Formulas II and III includes, for example, treating 6-alkoxy-8-quinazolinamine in pyridine with an alkyl halide. The reaction mixture can be heated to refluxing temperatures until there is no remaining starting material as evidenced by chromatography (e.g., TLC). The compound can then be purified by column chromatography to obtain 6-alkoxy-N-alkylquinazolin-8-amine, which can then undergo demethylation by treatment with HBr (48% aq) in a microwave initiator at 120° C. for 2-2.5 h. The reaction mixture can then be cooled to room temperature and the solvent can be evaporated using a Genevac DD-4X evaporator. The resulting residue can then be purified by silica gel chromatography to provide 6-alkoxy-N-alkylquinazolin-8-amine hydrobromide salt. The hydrobromide salt can be treated with a variety of halides in the presence of K₂CO₃ to provide the final 6,8-substituted quinazoline derivatives.

Compounds of Formula II can be prepared according to the method shown in the following scheme. In this example, A¹ is represented by Y, A² and A⁴ are CH, and A³ is N. Also in this example, A is (CH₂)₃, R⁸ and R⁹ are hydrogen, and R¹⁰ is substituted or unsubstituted amino A compound of Formula X wherein Ar is substituted quinozoline can also be synthesized according to this method.

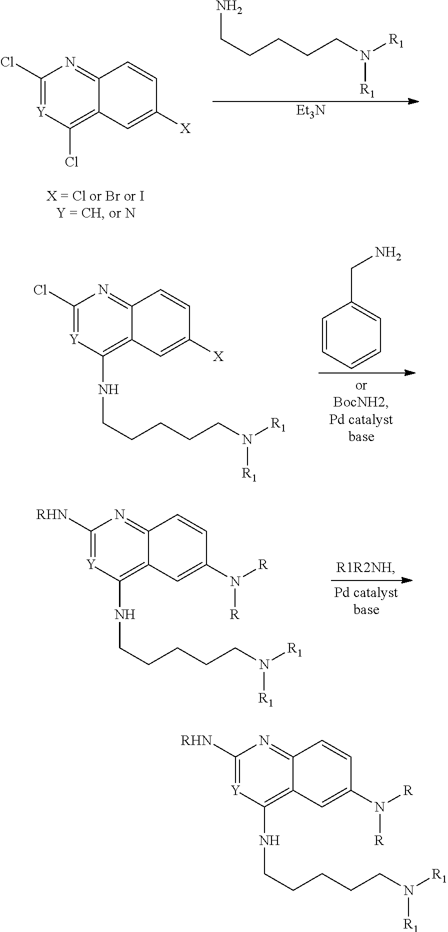

Compounds of Formula II-C can be prepared according to the method shown in the following scheme. In this example, A is (CH₂)₃, R⁸ and R⁹ are hydrogen, and R¹⁰ is substituted or unsubstituted amino A compound of Formula X wherein Ar is substituted naphthyridine can also be synthesized according to this method.

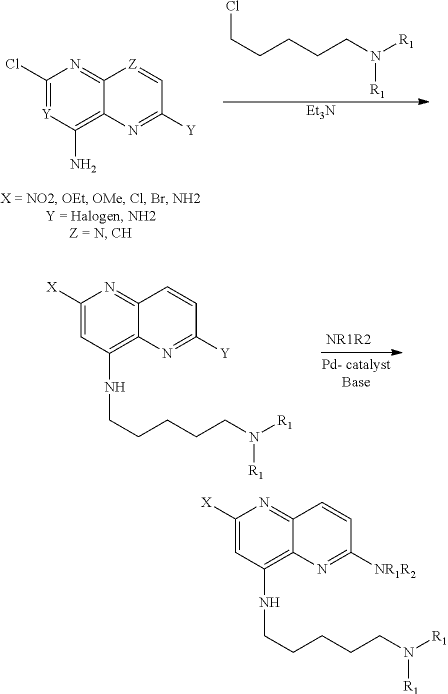

A general method of preparing a compound of Formula IV includes, for example, treating a 6-halo-4-nitro-1-napththonitrile with an amine, a palladium catalyst, and a base and then reducing the product using palladium on carbon. The resulting product can then be treated with an alkyl halide in triethylamine Subsequent treatment with base and hydrogen peroxide results in the desired compound. For example, a compound of Formula IV wherein A¹, A², and A⁴ are CH, A³ is CR⁷, R⁷ is substituted amino, R⁸ and R⁹ are hydrogen, R¹¹ is amido, and A is (CH₂)₃ can be synthesized according to the method shown below. A compound of Formula X wherein Ar is substituted naphthalene can also be synthesized according to this method.

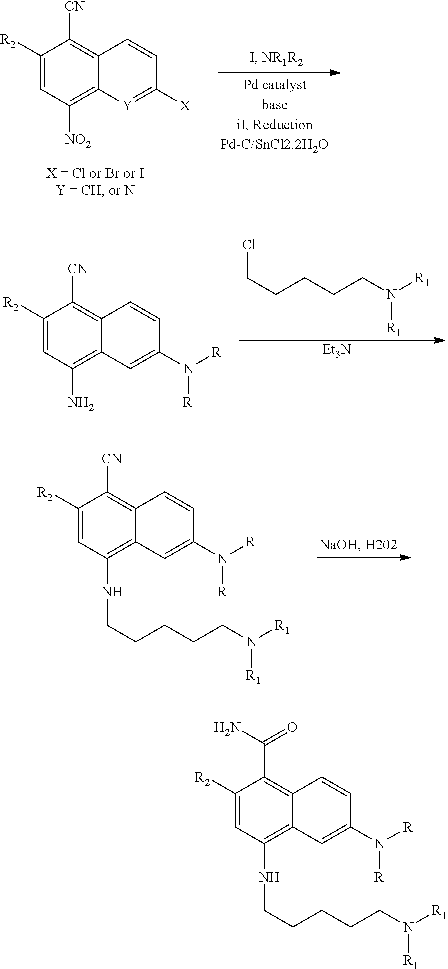

A general method of preparing a compound of Formula V includes, for example, stirring a mixture of 3,5-dinitropyridin-2-amine, 2-chlorophenol, and sodium acetate in a solvent mixture (e.g., EtOH and H₂O) under refluxing conditions. The solvent can be evaporated to provide a residue, which can be dissolved in H₂O and then extracted with an organic solvent (e.g., EtOAc). The organic layers can be combined and washed with H₂O and brine, dried over MgSO₄, and then concentrated in vacuo. Purification by column chromatography can provide 2-(3,5-dinitropyridin-2-ylamino)phenol. A mixture of the phenol compound and K₂CO₃ in solvent (e.g., DMF) can be stirred under refluxing conditions. The compound can be extracted with solvent (e.g., CH₂Cl₂), dried over MgSO₄, and purified by column chromatography to obtain 3-nitro-10H-benzo[e]pyrido[3,2-b][1,4]oxazine. The 3-nitro-oxazine can be treated with LHMDS in solvent (e.g., toluene). An alkyl halide can then be added and the reaction mixture heated (e.g., to 80° C.) until there is no remaining starting material as evidenced by chromatography (e.g., TLC). The crude compound can be purified by column chromatography to obtain 10-alkyl-3-nitro-10H-benzo[e]pyrido[3,2-b][1,4]oxazine, which can be subjected to hydrogenation to obtain amino-oxazine derivative. The amino-oxazine compound can then be treated with a variety of halides in the presence of pyridine or other bases to obtain substituted amines as the final products.

A general method of preparing a compound of Formulas VI, VII, VIII, and IX includes, for example, treating 7-chloro-5-nitro-1H-benzo[d]imidazole with an amine and K₂CO₃ under refluxing conditions in solvent, for example, CH₃CN. The reaction can then be quenched and the crude product can be purified by column chromatography to obtain N-alkyl-5-nitro-1H-benzo[d]imidazol-7-amine. The N-alkyl-5-nitro-1H-benzo[d]imidazol-7-amine can then be treated with a variety of halides in the presence of K₂CO₃ in solvent, for example, CH₃CN, to provide a 5-nitro-substituted imidazole. The imidazole can then be subjected to hydrogenation to afford a 5-amino-7-amine-substituted imidazole. Further treatment of the amine with alkyl halides can provide the final substituted amine.

Reactions to produce the compounds described herein can be carried out using the described or different solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The compounds described herein can be prepared using other methods known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are preferably suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

The term pharmaceutically acceptable salt as used herein refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught herein.)

The compounds described above or derivatives thereof are useful in treating cancer and other Hsp90 related diseases and conditions, such as inflammation and neurodegenerative disorders in humans, e.g., including pediatric and geriatric populations, and animals, e.g., veterinary applications. The methods described herein comprise administering to a subject a therapeutically effective amount of the compounds described herein or a pharmaceutically acceptable salt or prodrug thereof. Examples of Hsp90 related diseases include cancer and neurodegenerative disorders. The compounds described herein are also useful in treating inflammation associated with Hsp90 related diseases. As used herein, the terms promoting, treating, and treatment includes prevention; delay in onset; diminution, eradication, or delay in exacerbation of one or more signs or symptoms after onset; and prevention of relapse.

The methods and compounds as described herein are useful for both prophylactic and therapeutic treatment. For prophylactic use, a therapeutically effective amount of the compounds described herein or derivatives thereof are administered to a subject prior to onset (e.g., before obvious signs of an Hsp90 related disease), during early onset (e.g., upon initial signs and symptoms of an Hsp90 related disease), or an established Hsp90 related disease. Prophylactic administration can occur for several days to years prior to the manifestation of symptoms of the Hsp90 related disease. Prophylactic administration can be used, for example, in the preventative treatment of subjects diagnosed with genetic Hsp90 related disease. Therapeutic treatment involves administering to a subject a therapeutically effective amount of the compounds described herein or derivatives thereof after an Hsp90 related disease is diagnosed.

Administration of compounds described herein or derivatives thereof can be carried out using therapeutically effective amounts of the compounds described herein or derivatives thereof for periods of time effective to treat Hsp90 related diseases. The effective amount of the compounds described herein or derivatives thereof may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

In these methods, an Hsp90 related disease, for example, can be further treated with one or more additional agents. The one or more additional agents and the compounds described herein or derivatives thereof can be administered in any order, including simultaneous administration, as well as temporally spaced order of up to several days apart. The methods may also include more than a single administration of the one or more additional agents and/or the compounds described herein or derivatives thereof. The administration of the one or more additional agents and the compounds described herein or derivatives thereof may be by the same or different routes and concurrently or sequentially.

The examples below are intended to further illustrate certain aspects of the methods and compounds described herein, and are not intended to limit the scope of the claims.

### EXAMPLES

### Example 1

### Activity Characterization

Compound 1 was evaluated in a competitive binding assay (Du, Moulick et al. J. Biomol. Screen, 2007, 12, 915-24). The assay protocol and other assay protocols useful for the methods described herein are briefly described below.

### Hsp90 Binding FP Assay

To measure a therapeutically significant state of Hsp90, an FP assay that uses human cancer cell lysates (Du, Moulick et al. J. Biomol. Screen, 2007, 12, 915-24) was utilized. It probes the interaction of small molecules with tumor-specific Hsp90 and can therefore lead to inhibitors that are not only selective for cancer cells but also specific for a particular malignancy.

The dose-response effect of Compound 1 was tested in Hsp90 binding FP competition assays to obtain the IC₅₀ of the compound. The Hsp90 FP competition assay was performed in a 384-well black plate using the small-cell lung carcinoma (SCLC) cell line NCI-N417 as a source of tumor specific Hsp90 and the Cy3B-labeled geldanamycin (GM-cy3B) as the FP ligand. The compound was dissolved in DMSO and added at several concentrations to the reaction buffer containing both GM-cy3B (5 nM) and NCI-N417 cell lysate (1 mg/well) in a final volume of 50 μL. Free GM-cy3B (5 nM) and bound GM-cy3B with NCI-N417 cell lysate (5 nM GM-cy3B and 1 μg/well of NCI-N417 cell lysate) were included as controls in each plate. After incubating at room temperature for 2 to 16 hours, the polarization values were measured and expressed as millipolarization (mP) units using an Analyst HT plate reader (Molecular Devices). The competitive efficiency of Compound 1 was expressed as a percentage of control and calculated according to the following equation: % of control=((mPc−mPf)/(mPb−mPf))×100, where mPc is the recorded mP from wells containing compound, mPf is the average recorded mP from wells containing GM-cy3B-only, and mPb is the average recorded mP from wells containing both GM-cy3B and NCI-N417 lysate. IC₅₀ values were determined using a nonlinear regression analysis as implemented in Prism 4.0 (Graphpad Software).

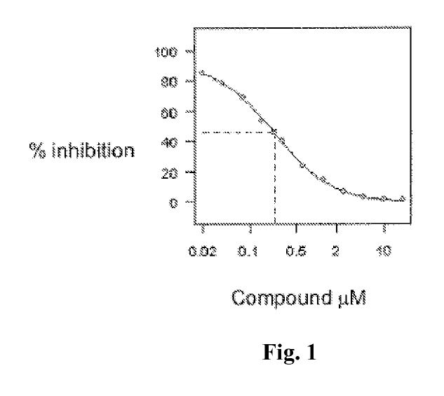

FIG. 1 is a graph showing the inhibition activity of compound 1 in a FP assay. The IC₅₀ of Compound 1 was determined as 200 nM.

### Cell-Based Her2 Degradation Assay by Western Blot

Hsp90 uniquely stabilizes the Her2/Hsp90 association (Xu, Mimnaugh et al. J. Biol. Chem. 2001, 276, 3702-3708). Addition of Hsp90 inhibitors to cancer cells induces the proteasomal degradation of a subset of proteins involved in signal transduction such as Raf1 kinase, Aid, and certain transmembrane tyrosine kinases such as Her2. Thus, Her2 degradation in cells is a functional read-out of Hsp90 inhibition. A correlation between Hsp90 binding and Her2 degradation in cancer cells is indicative of a selective biological effect in these cells via Hsp90.

A Western blot-based assay is used to measure the cellular level of Her2 protein in MCF-7 breast cancer cell lysates collected after 24 hours of compound treatment. MCF-7 cells are grown in RPMI-1640 medium supplemented with 10% fetal bovine serum (FBS). Then, 2×10⁵ cells are seeded in 24 well plates in 500 μL PRMI1640 medium and allowed to attach overnight. The compounds are dissolved in DMSO, added to the wells, and incubated for 24 hours. The cells are washed using ice-cold phosphate-buffered saline (PBS) and lysed in 1% NP40 lysis buffer (10 mM HEPES, pH 7.4, 150 mM NaCl, 1% Nonident P-40, 5 mM Pyrophosphate, 5 mM NaF, 2 mM Orthovanadate, 10 ug/ml Aprotinin, 10 ug/ml Leupeptin, 1 mM PMSF). After boiling with 6×SDS sample buffer, the cell lysates are separated by SDS-PAGE and transferred to nitrocellulose membrane. The membranes are blocked with 5% milk in tris-buffered saline buffer. The Her2 status is revealed by Western blotting with anti-Her2 antibody (Santa Cruz Biotechnology).

### Cell-Based Her2 Degradation Assay by Cytoblot

The cellular Her2 level after compound treatment is monitored by a microtiter-based Her2 degradation assay, which is the Cytoblot assay (Huezo, Vilenchik et al. Chem. Biol., 2003, 10, 629-634). This is a cell-based assay that enables the quantitative analysis of intracellular levels of Her2 protein.

The human breast cancer cell lines SKBr3 are maintained in RPMI1640 medium supplemented with 10% FBS. 3000 cells in 100 μL of growth medium are plated per well in black, clear-bottom microtiter plates (Corning) and allowed to attach for at least 24 hours at 37° C. and 5% CO₂. Compounds at different doses or the vehicle (DMSO) are carefully added to the wells and incubated for 24 hours. The cells are washed twice with ice-cold Tris buffer saline (TBS) containing 0.1% Tween 20 (TBST) and fixed with ice-cold methanol. After washing with TBST, the plate is incubated at RT for 1 hour with SuperBlock (Pierce 37535) and overnight at 4° C. with the anti-Her-2 antibody (Santa Cruz Biotechnology). The plate is then washed with TBST and incubated at RT for 2 hours with an anti-rabbit HRP-linked antibody (Sigma A0545). The chemiluminescent substrate solution (Pierce 38040) is added and the plate is read 5 minutes later in an Analyst HT plate reader (Molecular Devices). Luminescence readings resulting from compound-treated cells versus untreated cells (vehicle treated) are quantified and plotted against compound concentrations to give the IC₅₀ values (concentration of compound required to degrade 50% of total Her2).

### Cancer Cell Growth Inhibition Assay

The effects of the compounds on cancer cell growth are determined using the CellTiter-Blue cell viability assay (Promega). CellTiter-Blue® Cell Viability is based on the ability of living cells to convert a redox dye (resazurin) into a fluorescent end product (resorufin). Nonviable cells rapidly lose metabolic capacity and thus do not generate a fluorescent signal.

The SKBR3 breast cancer cells are plated in 384-well microtiter plates (Costar) and allowed to attach overnight. After treatment with either the compounds or the vehicle (DMSO) for 96 hours, the cells are measured for their viability by CellTiter-Blue. Briefly, 10 μL of CelTiter-Blue are added to each well in 384-well plates and incubated at 37° C. for 4 hours. The fluorescence intensity (FI) is measured using the Analyst HT plate reader (Molecular Devices) with an excitation at 545 nm and an emission at 595 nm. The IC₅₀ is calculated as the compound concentration that inhibits cell viability by 50% compared with vehicle control wells.

### Example 2

### Synthesis of Aminoquinoline Derivatives

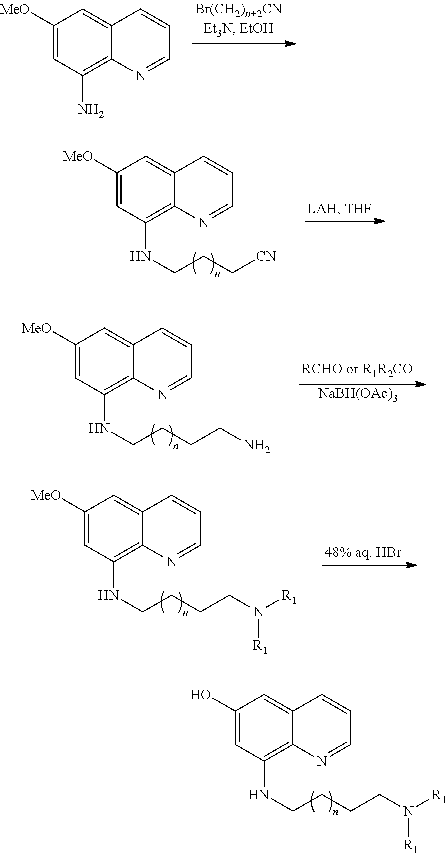

6-Methoxy-8-amino-quinoline (1.5 mmol, 261.3 mg) and 4-bromobutanenitrile (2.25 mmol, 333.0 mg) were refluxed in a mixture of Et₃N (2 ml) and MeOH (1 ml) for 24 hours. Additional 4-bromobutanenitrile (2.25 mmol, 333.0 mg) was added and the mixture was refluxed for another 48 hours. The mixture was concentrated to give a dark oily residue which was purified by chromatography using hexane/EtOAc (2:1) as the eluent to afford 4-(6-methoxyquinolin-8-ylamino)butanenitrile (148 mg, 40%). ¹H NMR (400 MHz, CDCl₃): δ 8.54 (dd, J=1.6 Hz, 4.0 Hz, 1H), 7.95 (dd, J=1.6 Hz, 8.4 Hz, 1H), 7.33 (dd, J=8.0 Hz, 4.4 Hz, 1H), 6.40 (d, J=2.8 Hz, 1H), 6.32 (d, J=2.8 Hz, 1H), 6.20 (m, 1H), 3.90 (s, 3H), 3.49 (q, J=6.4 Hz, 2H), 2.53 (t, J=7.2 Hz, 2H), 2.11 (quintet, J=6.8 Hz, 2H).

To a solution of 4-(6-methoxyquinolin-8-ylamino)butanenitrile (1.0 mmol, 1 equiv, 255.31 mg) in THF (5 ml) was added LAH (2.0 mmol, 2 equiv, 1.0 M in THF) dropwise at −78° C. The reaction mixture was kept at this temperature for an additional 2 hours, which was then allowed to slowly warm up to room temperature. The reaction was quenched by the addition of NaHCO₃ (satd aq) and extracted with EtOAc (3×5 ml). The combined organic layers were washed with brine and the resulting product was dried over Na₂SO₄. The solvent was evaporated to obtain the crude amine as an oily residue, which was subjected to the next step without further purification. To a solution of crude amine in 5 ml of CH₂Cl₂ at 0° C. was added acetaldehyde (85 IL, 1.52 mmol). The reaction mixture was kept at 0° C. for about 30 minutes, and then NaBH(OAc)₃ (350 mg, 1.65 mmol) was added to the above mixture. The resulting solution was allowed to warm to room temperature for another 3 hours. The mixture was diluted with CH₂Cl₂ (30 ml) and sequentially washed with 10% NaHCO₃ and brine. Drying the organic layers over sodium sulfate followed by filtration and concentration provided a residue that was purified by silica gel chromatography to obtain N¹,N¹-diethyl-N⁴-(6-methoxyquinolin-8-yl)butane-1,4-diamine (90 mg, 43%). ¹H NMR (400 MHz, MeOH-d₄): δ 8.61 (dd, J=3.9, 1.6 Hz, 1H), 8.13 (dd, J=8.6, 1.6 Hz, 1H), 7.36 (dd, J=8.6, 3.9 Hz, 1H), 6.86 (d, J=2.3 Hz, 1H), 6.83 (d, J=2.3 Hz, 1H), 3.88 (s, 3H), 3.37 (quartet, J=7.0 Hz, 2H), 2.47 (quartet, J=7.0 Hz, 4H), 2.35 (t, J=7.8 Hz, 2H), 1.55-1.48 (m, 2H), 1.41-1.33 (m, 2H), 1.26-1.21 (m, 2H), 0.96 (t, J=7.0 Hz, 6H). MS, m/z (C₁₉H₂₉N₃O): calcd, 315.2; found, 316.4 (MH).

A solution of N¹,N¹-Diethyl-N⁴-(6-methoxyquinolin-8-yl)butane-1,4-diamine (10 mg, 0.031 mmol) in HBr (1 ml, 48% aq) was heated to 120° C. in a microwave initiator for 2.5 h. The reaction mixture was cooled and the solvent was then evaporated using a Genevac DD-4X evaporator. The resulting dark brown residue was purified by silica gel chromatography to provide 8-(5-(diethylamino)pentylamino)quinolin-6-ol (8 mg, 55%) as a pale brown solid. ¹H NMR (400 MHz, DMSO-d₆): δ 9.12 (br s, 1H), 8.60 (d, J=3.9 Hz, 1H), 8.30 (d, J=7.8 Hz, 1H), 7.56 (dd, J=7.8, 4.6 Hz, 1H), 6.50 (s, 1H), 6.43 (s, 1H), 3.21 (t, J=7.0 Hz, 2H), 3.08 (quintet, J=7.0 Hz, 4H), 3.03-2.98 (m, 2H), 1.74-1.62 (m, 4H), 1.47-1.39 (m, 2H), 1.16 (t, J=7.0 Hz, 6H). ¹³C NMR (100 MHz, DMSO-d₆): δ 158.5, 143.9, 142.0, 133.0, 132.9, 130.7, 122.5, 100.4, 92.4, 54.5, 51.3, 46.8, 28.1, 24.4, 23.5, 9.2, 9.1. EI, m/z (C₁₈H₂₇N₃O): calcd, 301.4; found: 302.3 (M+H). Anal. Calcd for C₁₈H₂₉Br₂N₃O.H₂O: C, 44.92; H, 6.49; N, 8.73; observed: C, 45.50; H, 6.26; N, 8.59.

### Example 3

### Synthesis of Quinazoline Derivatives

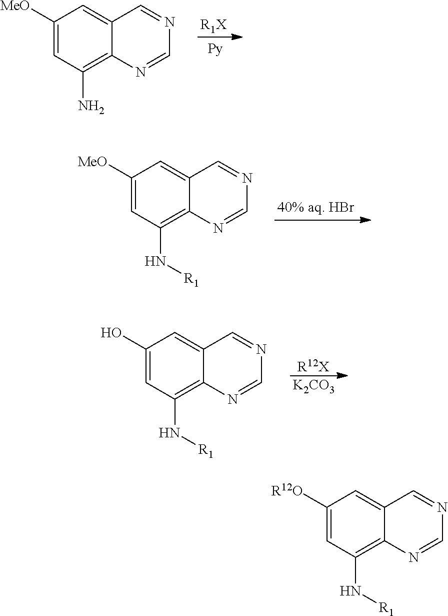

6-Methoxy-8-quinazolinamine (1.0 mmol) in pyridine (5 ml) was treated with R₁X (1.1 mmol). The reaction mixture was heated to refluxing temperatures until there was no remaining starting material as evidenced by chromatography (e.g., TLC). The compound was purified by column chromatography to obtain 6-methoxy-N-alkylquinazolin-8-amine, which was then de-methylated by treatment with HBr (1 ml, 48% aq) in a microwave initiator at 120° C. for 2-2.5 h. The reaction mixture was cooled to room temperature and the solvent was then evaporated using a Genevac DD-4X evaporator. The resulting dark brown residue was purified by silica gel chromatography to provide 6-hydroxy-N-alkylquinazolin-8-amine hydrobromide salt. The hydrobromide salt was then treated with a variety of halides in the presence of K₂CO₃ to provide the final 6,8-substituted quinazoline derivatives.

### Example 4

### Synthesis of Amino-Oxazine Derivatives

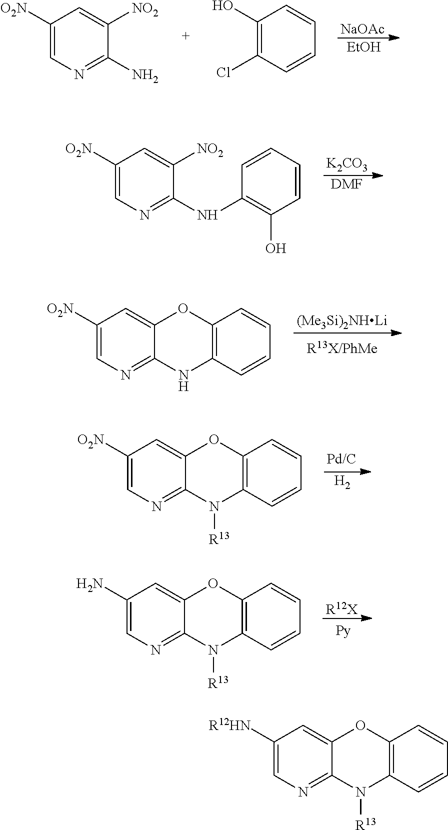

A mixture of 3,5-dinitropyridin-2-amine (1.0 mmol), 2-chlorophenol (1.0 mmol) and sodium acetate (4.0 mmol) in EtOH (4 ml) and H₂O (1 ml) was stirred under refluxing conditions for 24 h. The solvent was evaporated to provide a residue, which was dissolved in H₂O and extracted with EtOAc. The organic layers were combined and washed with H₂O and brine, dried over MgSO₄, and then concentrated in vacuo. Purification by column chromatography provided 2-(3,5-dinitropyridin-2-ylamino)phenol.

A mixture of the phenol compound (0.5 mmol) and K₂CO₃ (0.6 mmol) in DMF was stirred under reflux for 12 h. The compound was extracted with CH₂Cl₂ (3×8 ml), dried over MgSO₄, and purified by column chromatography to obtain 3-nitro-10H-benzo[e]pyrido[3,2-b][1,4]oxazine.

The 3-nitro-oxazine (0.1 mmol) was treated with LHMDS (1M in hexanes) in toluene for 20 min. An alkyl halide was then added and the reaction mixture was kept at 80° C. until there was no remaining starting material as evidenced by chromatography (e.g., TLC). The crude compound was purified by column chromatography to obtain 10-alkyl-3-nitro-10H-benzo[e]pyrido[3,2-b][1,4]oxazine, which was subjected to hydrogenation to obtain amino-oxazine derivative.

The amino-oxazine compound was then treated with a variety of halides in the presence of pyridine or other bases to obtain substituted amines as the final products.

### Example 5

### Synthesis of Amino-Imidazole Derivatives

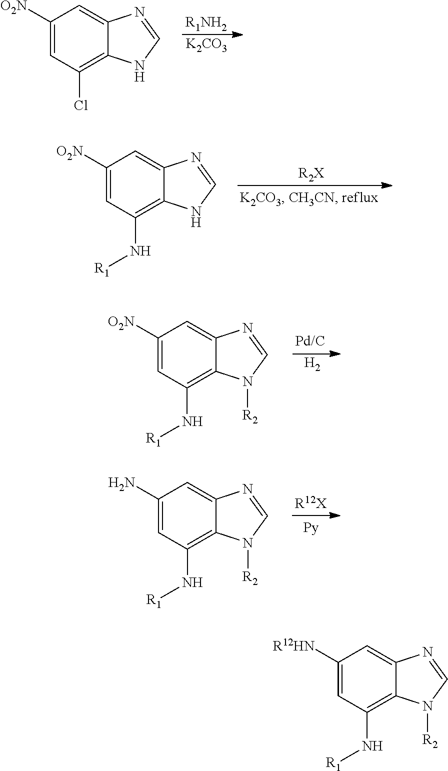

7-Chloro-5-nitro-1H-benzo[d]imidazole (1.0 mmol), an amine (1.1 mmol) and K₂CO₃ were refluxed in CH₃CN (5 mL) for 12 h. The reaction was quenched and the product was purified by column chromatography to obtain N-alkyl-5-nitro-1H-benzo[d]imidazol-7-amine N-alkyl-5-nitro-1H-benzo[d]imidazol-7-amine was then treated with a variety of halides in the presence of K₂CO₃ in CH₃CN to provide 5-nitro-substituted imidazole. The imidazole was then subjected to hydrogenation to afford 5-amino-7-amine-substituted imidazole. Further treatment of the amine with alkyl halides provided the final substituted amine.

A number of embodiments have been described. Nevertheless, it will be understood to one skilled in the art that various modifications may be made. Further, while only certain representative combinations of the formulations, methods, or products are disclosed herein are specifically described, other combinations of the method steps or combinations of elements of a composition or product are intended to fall within the scope of the appended claims. Thus a combination of steps, elements, or components may be explicitly mentioned herein; however, all other combinations of steps, elements, and components are included, even though not explicitly stated.

1. A compound of the following formula: 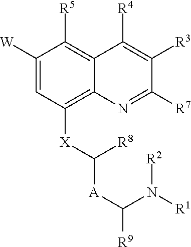
or a pharmaceutically acceptable salt or prodrug thereof, wherein:
A is substituted or unsubstituted C₁₋₆ alkyl, substituted or unsubstituted C₂₋₆ alkenyl, or substituted or unsubstituted C₂₋₆ alkynyl;
W is NHR⁶, OR⁶, or hydrogen;
X is NH, S, or O;
R¹ and R² are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl, wherein R¹ and R² together are substituted or unsubstituted C₁₋₈ alkyl or substituted or unsubstituted C₂₋₈ alkenyl;
R³, R⁴, and R⁵ are each independently selected from hydrogen, halogen, hydroxyl, nitro, cyano, substituted or unsubstituted amino, substituted or unsubstituted thio, substituted or unsubstituted C₁₋₁₂ alkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted C₁₋₁₂ alkylamine, substituted or unsubstituted C₂₋₁₂ alkenylamine, substituted or unsubstituted C₂₋₁₂ alkynylamine, substituted or unsubstituted arylamine, substituted or unsubstituted acyl, substituted or unsubstituted alkoxyl, and substituted or unsubstituted aryloxyl;
R⁶ is hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted acyl, substituted or unsubstituted aminoalkyl, substituted or unsubstituted hydroxyalkyl, or substituted or unsubstituted alkoxyalkyl;
R⁷ is hydrogen, hydroxyl, substituted or unsubstituted alkoxyl, substituted or unsubstituted amino, substituted or unsubstituted thio, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, or substituted or unsubstituted sulfonyl; and
R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl,
wherein if X is NH, W is hydroxyl or methoxyl, and one of R¹ or R² is C₁₋₅ alkyl, the other of R¹ or R² is not hydrogen or C₁₋₅ alkyl;
if X is NH and W is hydroxyl or methoxyl, R¹ and R² are not simultaneously hydrogen; and
if X is NH, W is hydroxyl or methoxyl, and R⁸ is methyl, R⁹ is not hydrogen.

2. The compound of claim 1, wherein X is NH.

3. The compound of claim 1, wherein R⁵ is substituted or unsubstituted amido.

4. The compound of claim 1, wherein R⁷ is substituted or unsubstituted amino.

5. The compound of claim 1, wherein the compound is

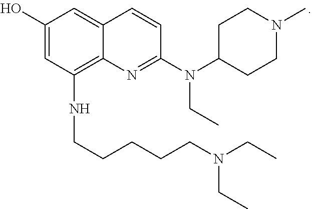

6. The compound of claim 1, wherein the compound has the following formula: 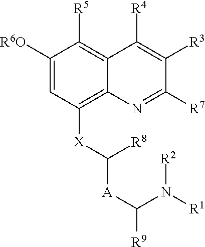
or a pharmaceutically acceptable salt or prodrug thereof, wherein:
A is substituted or unsubstituted C₁₋₆ alkyl, substituted or unsubstituted C₂₋₆ alkenyl, or substituted or unsubstituted C₂₋₆ alkynyl;
X is S or O;
R¹, R², and R⁷ are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl, wherein R¹ and R² together are substituted or unsubstituted C₁₋₈ alkyl or substituted or unsubstituted C₂₋₈ alkenyl;
R³, R⁴, and R⁵ are each independently selected from hydrogen, halogen, hydroxyl, nitro, substituted or unsubstituted amino, cyano, substituted or unsubstituted C₁₋₁₂ alkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted C₁₋₁₂ alkylamine, substituted or unsubstituted C₂₋₁₂ alkenylamine, substituted or unsubstituted C₂₋₁₂ alkynylamine, substituted or unsubstituted arylamine, substituted or unsubstituted alkoxyl, and substituted or unsubstituted aryloxyl;
R⁶ is hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted acyl, substituted or unsubstituted aminoalkyl, substituted or unsubstituted hydroxyalkyl, or substituted or unsubstituted alkoxyalkyl; and
R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl.

7. The compound of claim 6, wherein R¹ is hydrogen, methyl, ethyl, propyl, butyl, pentyl, isopropyl, sec-butyl, isobutyl, benzyl, benzoyl, acetyl, or substituted or unsubstituted sulfonyl.

8. The compound of claim 6, wherein R² is hydrogen, methyl, ethyl, propyl, butyl, pentyl, isopropyl, sec-butyl, isobutyl, benzyl, benzoyl, acetyl, or

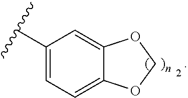

9. The compound of claim 6, wherein R¹—N—R² combine to form aziridine, azetidine, pyrrolidine, or piperidine.

10. The compound of claim 6, wherein R³ is hydrogen, methyl, ethyl, propyl, or NH-ethyl.

11. The compound of claim 6, wherein R⁴ is hydrogen, methyl, ethyl, propyl, NH-ethyl, NH-phenyl, chloro, hydroxyl, methoxyl, or phenoxyl.

12. The compound of claim 6, wherein R⁵ is hydrogen, hydroxyl, chloro, nitro, amino, NH-phenyl, p-chlorophenyl, or p-methoxyphenyl.

13. The compound of claim 6, wherein R⁶ is hydrogen, methyl, ethyl, allyl, acetyl, or —(CH₂)n₁T, wherein n₁ is 1-5 and T is hydrogen, hydroxyl, amino, or alkoxyl.

14. The compound of claim 6, wherein R⁷ is hydrogen, methyl, ethyl, propyl, isopropyl, butyl, isobutyl, t-butyl, acetyl, or propanoyl.

15. The compound of claim 6, wherein R⁸ is hydrogen, methyl, or ethyl.

16. The compound of claim 6, wherein R⁹ is hydrogen, methyl, or ethyl.

17-68. (canceled)

68. A composition comprising a compound of claim 1 and a pharmaceutically acceptable carrier.

69. A method preventing or treating cancer in a subject comprising administering to the subject a therapeutically effective amount of the compound or compositions of claim 1.

70. A method of preventing or treating Hsp90 related diseases in a subject comprising administering to the subject a therapeutically effective amount of the compound or compositions of claim 1.

71-72. (canceled)

73. A method of preventing or treating Hsp90 related diseases in a subject comprising selecting a subject with a Hsp90 related disease and administering to the subject a therapeutically effective amount of the compound or compositions of the following formula: 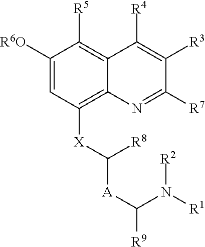
or a pharmaceutically acceptable salt or prodrug thereof, wherein:
A is substituted or unsubstituted C₁₋₆ alkyl, substituted or unsubstituted C₁₋₆ haloalkyl, substituted or unsubstituted C₂₋₆ alkenyl, or substituted or unsubstituted C₂₋₆ alkynyl;
X is NH, S, or O;
R¹, R², and R⁷ are each independently selected from hydrogen, substituted or unsubstituted C₁₋₁₂ alkyl, substituted or unsubstituted C₁₋₁₂ haloalkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl, substituted or unsubstituted acyl, and substituted or unsubstituted sulfonyl, wherein R¹ and R² together are substituted or unsubstituted C₁₋₈ alkyl or substituted or unsubstituted C₂₋₈ alkenyl;
R³, R⁴, and R⁵ are each independently selected from hydrogen, halogen, nitro, substituted or unsubstituted amino, cyano, substituted or unsubstituted C₁₋₁₂ alkyl, substituted or unsubstituted C₂₋₁₂ alkenyl, substituted or unsubstituted C₂₋₁₂ alkynyl, substituted or unsubstituted aryl, substituted or unsubstituted cycloalkyl, substituted or unsubstituted heteroaryl, substituted or unsubstituted heterocycloalkyl, substituted or unsubstituted arylalkyl, substituted or unsubstituted heteroarylalkyl, substituted or unsubstituted cycloalkylalkyl, substituted or unsubstituted heterocycloalkylalkyl substituted or unsubstituted C₁₋₁₂ alkylamine, substituted or unsubstituted C₂₋₁₂ alkenylamine, substituted or unsubstituted C₂₋₁₂ alkynylamine, substituted or unsubstituted arylamine, substituted or unsubstituted alkoxyl, and substituted or unsubstituted aryloxyl;
R⁶ is hydrogen, halogen, substituted or unsubstituted C₁₋₁₂ alkyl, substituted or unsubstituted C₂₋₁₂ alkenyl, and substituted or unsubstituted C₂₋₁₂ alkynyl, substituted or unsubstituted acyl, substituted or unsubstituted aminoalkyl, hydroxyalkyl, or substituted or unsubstituted alkoxyalkyl; and
R⁸ and R⁹ are each independently selected from hydrogen, substituted or unsubstituted C₁₋₁₂ alkyl, substituted or unsubstituted C₂₋₁₂ alkenyl, and substituted or unsubstituted C₂₋₁₂ alkynyl,
wherein if X is NH, R⁶ is hydrogen or methyl, and one of R¹ or R² is C₁₋₅ alkyl, the other of R¹ or R² is not hydrogen or C₁₋₅ alkyl;
if X is NH and R⁶ is hydrogen or methyl, R¹ and R² are not simultaneously hydrogen; and
if X is NH, R⁶ is hydrogen or methyl, and R⁸ is methyl, R⁹ is not hydrogen.

74-77. (canceled)
